(19) United States
(12) Patent Application Publication
(10) Pub. No.: US 2023/0107712 A1
(43) Pub. Date: Apr. 6, 2023

(54) System, Method And Computer Program Product Which Uses Biometrics As A Feedback For Home Control Monitoring To Enhance Wellbeing

(71) Applicant: VEEV GROUP, INC., San Mateo, CA (US)

(72) Inventors: Binyamin GIL, Rehovot (IL); Mati COHEN, Ganei Tikva (IL); Israel Jay KLEIN, Kfar Saba (IL)

(21) Appl. No.: 17/905,971

(22) PCT Filed: Mar. 24, 2021
(86) PCT No.: PCT/IL2021/050332

§ 371 Date: Sep. 9, 2022

Related U.S. Application Data
(60) Provisional application No. 62/993,734, filed on Mar. 24, 2020.

(51) Int. Cl. G05B 19/042

(52) U.S. Cl. CPC G05B 19/042; G05B 2219/2642

(57) ABSTRACT

A smart home control method comprising using at least one hardware processor to perform the following: generating an initial set of recommendations for, and/or operative limitations on, home control actions; measuring wellbeing indexes aka scores and using the scores as feedback to determine which home control actions improve the scores including introducing perturbations of device operation including applying at least one perturbation to at least one parameter of at least one home appliance; measuring the perturbations' effect on the scores, and further optimizing of house management to yield increased wellbeing by retaining post-perturbation values of at least one individual parameter of at least one individual home appliance, which yielded increased wellbeing relative to pre-perturbation values of the individual parameter of the individual home appliance.

### REFERENCE TO CO-PENDING APPLICATIONS

Priority is claimed from U.S. Provisional Patent Application No. 62/993,734, entitled “System, Method And Computer Program Product Which Uses Biometrics As A Feedback For Home Control Monitoring . . . ” and filed 24 Mar. 2020, the disclosure of which application/s is hereby incorporated by reference.

### FIELD OF THIS DISCLOSURE

The present invention relates generally to computerized systems, and more particularly to computerized systems for controlling devices such as household appliances.

### BACKGROUND FOR THIS DISCLOSURE

Impact of smart homes is described by Wikipedia https://en.wikipedia.org/wiki/Home_automation#Applications_and_technologies as including only 2 impacts—energy saving, and enhanced security while the homeowner is away:

“Utilizing home automation could lead to more efficient and intelligent energy saving techniques . . . researchers propose using data from sensors regarding consumer activity within the home to anticipate the consumer needs and balance that with energy consumption . . . . Furthermore, home automation has large potential regarding family safety and security . . . . Home automation includes . . . smart security systems and surveillance setups. This allows consumers to monitor their homes while away, and to give trusted family members access to that information in case anything bad happens.”

Specifically for the elderly and disabled, Wikipedia describes https://en.wikipedia.org/wiki/Home_automation_for_the_elderly_and_disabled that:

“There are two basic forms of home automation systems for the elderly: embedded health systems and private health networks.

Embedded health systems integrate sensors and microprocessors in appliances, furniture, and clothing, which collect data that is analyzed and can be used to diagnose diseases and recognize risk patterns. Private health networks implement wireless technology to connect portable devices and store data in a household health database. Due to the need for more healthcare options for the aging population “there is a significant interest from industry and policy makers in developing these technologies.

Home automation is implemented in homes of older adults and people with disabilities in order to maintain their independence and safety, also saving the costs and anxiety of moving to a health care facility. For the disabled smart homes give them opportunity for independence, providing emergency assistance systems, security features, fall prevention, automated timers, and alerts, also allowing monitoring from family members via an internet connection . . . . Some of the monitoring or safety devices that can be installed in a home include lighting and motion sensors, environmental controls, video cameras, automated timers, emergency assistance systems, and alerts . . . .

Home automation systems may include automatic reminder systems for the elderly. Such systems are connected to the Internet and make announcements over an intercom. They can prompt about . . . turning off the stove, closing the blinds, locking doors, etc. Users choose what activities to be reminded of. The system can be set up to automatically perform tasks based on user activity, such as turning on the lights or adjusting room temperature when the user enters specified areas . . . . Domestic robots, connected to the domotic network, are included to perform or help in household chores for example: Rotimatic which makes rotis, tortillas and puris from scratch.” (see https://en.wikipedia.org/wiki/Home_automation_for_the_elderly_and_disabled).

Wikipedia points out that “tailoring home automation toward the elderly has generated opposition. It has been stated that “Smart home technology will be helpful only if it is tailored to meet the individual needs of each patient” . . . many of the interfaces designed for home automation “are not designed to take functional limitations, associated with age, into consideration”. Another presented problem involves . . . the elderly who often have difficulty operating electronic devices. The cost of the systems has also presented a challenge, as the U.S. government currently provides no assistance to seniors who choose to install these systems.”

There are substantial positive associations between health and subjective wellbeing. Those who rate their general health as “good” or “excellent” tend to describe better subjective wellbeing compared to those who rate their health as “fair” or “poor”.

Quality of sleep is an important indicator of health and wellbeing. Heart rate is a significant indicator of heart and blood vessels' general condition. Both parameters could be measured passively, without contact, and without interfering with the subject's normal life at home.

Traditional methods to assess wellbeing use questionnaires that are subjective, and thus biased.

Perturbation theory is known and is described e.g. by Wikipedia, here: https://en.wikipedia.org/wiki/Perturbation_theory#Terminology. This theory studies deviations of moving objects or processes from their regular state or path, caused by an outside influence. For example, the regular course of motion of a celestial body, produced by some force F1, may be elliptical, however an additional force F2 may produce a perturbation relative to the normal elliptical course.

Perturbation technique is a computational technique to eliminate linear terms in an equation, and retain only nonlinear terms. Physiotherapists also speak of introducing perturbations so as to teach balance, for example, by pushing a patient slightly so the patient can practice maintaining balance.

US20140078061 teaches introducing unexpected and subtle perturbations (e.g., small changes in mouse velocity, position and/or acceleration) in the response of a computer mouse, and measuring the motor responses of the individual user.

Introducing perturbations is used as a paradigm to investigate learning e.g. as described here: https://journals.plos.org/plosone/article?id=10.1371/journal.pone.0220884.

Perturbation-based methods are used in machine learning to augment optimization techniques e.g. as described here (according to the online summary): https://mitpress.mit.edu/books/perturbations-optimization-and-statistics.

Matlab's perturb function applies perturbations to objects e.g. as described here: https://www.mathworks.com/help/nav/ref/inssensor.perturb.html:isessionid=5cac2db19665b232a25ffc57a45b.

Activity trackers, aka fitness trackers, are known. Some of these devices monitor or track heartbeat. These wearables may be synced, e.g. wirelessly, to a computer or smartphone which provides long-term data tracking.

Fitbit, Inc. distributes activity trackers, smartwatches, and wireless-enabled wearable technology devices. These devices measure fitness metrics such as number of steps walked, heart rate, quality of sleep, number of steps climbed. Wikipedia reports that “In 2019, Google announced its intention to buy Fitbit for $2.1 billion. The transaction was completed in January 2021 . . . Fitbit reports to have sold more than 100 million devices and have 28 million users”. Nonetheless, Wikipedia goes on to say, “While these devices appear to increase physical activities, there is little evidence that they improve health outcomes”.

It is known that “lack of sufficient exposure to high-intensity light during the morning can negatively affect the health and well-being of residents in dementia care facilities” (https://www.sciencedirect.com/science/article/abs/pii/S0360132318301367).

An open-access circadian stimulus (CS) calculator is described and available online e.g here: https://www.lrc.rpi.edu/resources/newsroom/pr_story.asp?id=338#.YD9q2mgzZPY. The CS calculator converts photopic illuminance provided by a given light source operating at a given light level during a one-hour exposure time window, into a metric expressing that light's effectiveness for stimulating the human circadian system, based on an RPI model of “how the retina converts light signals into neural signals for the circadian system”. The developers recommend their work as being able “to help lighting professionals select light sources and light levels . . . in architectural spaces” using “lighting design objectives that differ from those typically used in traditional architectural lighting design”.

The disclosures of all publications and patent documents mentioned in the specification, and of the publications and patent documents cited therein directly or indirectly, are hereby incorporated by reference other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one hardware processor in communication with at least one memory, with instructions stored in such memory executed by the processor to provide functionalities which are described herein in detail. Any functionality described herein may be firmware-implemented or processor-implemented, as appropriate.

Certain embodiments seek to provide reliable empirical information regarding wellbeing or health which would better serve wellbeing related decision making.

In certain embodiments herein, data retrieved from multiple sensors, distributed in the house (e.g. any of the environmental sensors described herein), are used to measure biometrics from which a wellbeing score and/or health indexes are computed in an objective, quantitative and reproducible manner (see e.g. using data source 2i).

Based on scientific published literature (see e.g. using data source 2e), a set of recommendations for, and actual modification of, home devices control are derived to facilitate optimal comfort and wellbeing. For example, high light intensity during the evening can interfere with the biological clock hormone cycle, thus reducing sleep quality. A set of recommended light intensity levels could be prompted to the user during different times of the day in order to prevent such interference. A modification may be implemented by reducing light intensity by 20% after 7 pm, for example. These recommendations/modifications may improve sleep quality, and therefore health and wellbeing of the user. Wellbeing score is used to monitor the wellbeing of the user and as a feedback that validates that the system operates indeed to the benefit of the user. Alternatively or in addition, minor perturbations of device operation (sometimes seamless to the user) may be introduced e.g. as illustrated in FIG. 1, and their effect on the wellbeing score may be measured in order to allow further optimization of the house management for greater wellbeing effect. Perturbations, modifications and recommendations could be in short time intervals (minute scale) or longer (hours and days), depending on the device and/or the controlled parameter and/or the wellbeing effector considered.

This method facilitates evidence-based design and real home automatic operation, unlike current available home automation systems. For example, in current “smart home” systems, the user activity (device operation) is recorded and analyzed, and the system uses AI to learn and mimic the user activity by inferring the next step/operation that may be done by the user and perform it automatically—regardless of the wellbeing effect of the operation. In the proposed system, wellbeing effect is the main factor by which future operations are modified, initially according to state of the art public knowledge about general wellbeing effect, and later by measuring the actual effect on the user's wellbeing, which may be different from the effect on the general population.

Another advantage is that the system database can move with the user from one house to the other, therefore preserving the unique (personal) wellbeing affecting factors.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by, server A. Analogously, the remote processor P may not, itself, perform all of the operations, and, instead, the remote processor P itself may receive output/s of portion/s of the operation from yet another processor/s P′, may be deployed off-shore relative to P, or “on a cloud”, and so forth.

The present invention thus typically includes at least the following embodiments:

Embodiment A1. A smart home control method or system or computer program product comprising generating an initial set of recommendations for, and/or operative limitations on, home control actions; and/or measuring wellbeing and health indexes (or scores or parameters) and using the indexes as feedback to validate that the home control actions improved the indexes including introducing minor perturbations of device operation and/or measuring the perturbations' effect on the indexes, which typically allows further optimization of house management to yield greater wellbeing or health effect.

Embodiment A2. A method or system or computer program product according to any of the preceding embodiments which stores a matrix of home devices controllable parameters, wherein a threshold level (e.g., representing a numerical value representing the maximum absolute difference from a preset optimal value) is given for each parameter to decide if perturbation or user recommendation may be used to adjust the optimal device value.

Embodiment A3. A method or system or computer program product e.g. any of the preceding embodiments wherein for each home devices controllable parameters, a matrix of S×T is given where S is a scene in which the device can take part in and T is a time of the day and wherein each vector in the matrix contains an optimal/recommended value.

Embodiment A4. A method or system or computer program product according to any of the preceding embodiments wherein initial optimal/recommended values are given based on data from the literature whereas, upon use and optimization process, the values may be changed to allow personalization.

Embodiment A5. A method or system or computer program product e.g. according to any of the preceding embodiments which executes a classification algorithm to classify Tenant state.

For example, a tenant “sick” state may be either “ill” or “healthy” and a suitable classifier is employed for differentiating between these 2 possible states. The classifier can be some simple decision based on measurements of body temperature or heart rate. For example, if (body temperature>T) and (average heart rate>R) then the tenant state is set to “ill” otherwise set to “healthy”. In this case, T and R are some threshold values which are preset based on general population averages but can be updated over time as the individual nature is unique (e.g., mean heart rate of athletes tend to be lower than the general population average). Over time a more complex classifier could be employed as additional variable measurements may be taken and correlated with the target decision state (i.e., measuring sleep duration). In addition, these simple classifiers may be used to label other data analysis processes. For example, the energy consumption metrics of the house may be labeled or tagged by the tenant states (e.g., number of tenants which are “ill”).

Embodiment A6. A method or system or computer program product according to any of the preceding embodiments wherein classes of tenant state include at least one of: sleep, active, resting.

Embodiment A7. A method or system or computer program product according to any of the preceding embodiments wherein, for at least one given state, indexes relevant for that state are computed.

Embodiment A8. A method or system or computer program product according to any of the preceding embodiments wherein for a given scene/time combination, the parameters are sorted in decreasing difference order and, at least once, a value with a next highest absolute difference (AD) from the optimum is selected.

Embodiment A9. A method or system or computer program product according to any of the preceding embodiments wherein at least once, an optimal value is proposed to a tenant when the given scene/time combination next recurs.

Embodiment A10. A method or system or computer program product according to any of the preceding embodiments wherein the optimal value is proposed if absolute difference (AD)>Mth.

Embodiment A11. A method or system or computer program product according to any of the preceding embodiments, wherein at least once, a perturbation of the value towards the optimum is introduced, when the given scene/time combination next recurs.

Embodiment A12. A method or system or computer program product according to any of the preceding embodiments wherein the perturbation is introduced if absolute difference (AD)<Mth.

Embodiment A13. A method or system or computer program product according to any of the preceding embodiments wherein a penalty procedure is used, including moving to a next parameter on a list of parameters sorted in decreasing difference order, if a tenant reverses an automatic perturbation.

Embodiment A14. A method or system or computer program product according to any of the preceding embodiments wherein a smoothing procedure is used in which, for every parameter changed, after validation of wellbeing effect,—an adjacent time interval is changed to apply change to scenes close in time in the S×T matrix thereby to initiate optimization of rarely measured scenes.

Embodiment A15. A method or system or computer program product according to any of the preceding embodiments wherein Monte Carlo method is used to produce randomness thereby to avoid local minimum traps.

Embodiment A16. An automated system for optimizing quality of life of tenants, the system comprising all or any subset of the following: wellbeing enhancement logic, stored in computer memory, which defines how appliances' modes of operations affect at least one end-user's wellbeing at at least one time t; and/or a hardware processor (aka wellbeing processor) in data communication with the logic; and/or sensors which are configured to generate at least one measurement of at least one aspect of an end-user's wellbeing and to feed the measurement to the hardware processor; and/or smart home apparatus including at least one controller which controls at least one digitally controlled home appliance. Typically, the hardware processor is configured to command the at least one controller to at least once control the at least one digital appliance to transit from a first mode of operation to a second mode of operation at a time t, wherein the second mode of operation serves the end-user's wellbeing at time t better than the first mode of operation does, according to the wellbeing enhancement logic.

It is appreciated that the wellbeing enhancement logic may be stored in computer memory in any suitable format e.g. as rules. For example, one rule which defines how appliances' modes of operations affect at least one end-user's wellbeing at at least one time t may be that blue light adversely affects wellbeing at a certain time of day t1 but increases wellbeing at another time of day t2. Or, another rule may define that a certain temperature increases wellbeing at any time of day in which a user is active, whereas another, high temperature value increases wellbeing at any time of day in which the user is sleeping.

### EXAMPLES

a. Digital appliance is at least one light fixture. First mode of operation is provision of normal daytime light; time t=evening; second mode of operation is provision of illumination with less blue light (possibly by reducing the intensity of all wavelengths of light by, say, 20%). Or, digital appliances are all light fixtures in the house; first mode of operation is a light fixture control schedule which either is controlled entirely manually, or is controlled automatically without regard to Circadian needs, and the second mode of operation is a light fixture control schedule which is controlled at least partly automatically with regard to Circadian needs e.g. as described herein. any suitable technology may be employed to reduce blue light at certain times in the day e.g. during the evening e.g. as described here—https://www.webmd.com/sleep-disorders/sleep-blue-light.

b. digital appliance is an air conditioner. First mode of operation is provision of room temperature of about 25 degrees; time t=a time window during which a senior is known to be exercising, second mode of operation is provision of room temperature of about 20 degrees.

c. digital appliance is window shades. First mode of operation is a certain degree of open-ness of the shades e.g. half-open. Time t=a time at which the reader is known to be reading. Second mode of operation is a different degree of open-ness of the shades which optimizes light intensity and/or temperature or thermal comfort, for reading.

Embodiment A17. A system according to any of the preceding embodiments wherein the home appliance comprises a light fixture, shades, an air conditioner, a humidifier, or a window.

Embodiment A18. A system according to any of the preceding embodiments wherein at least one of the sensors is wearable by the end-user.

Embodiment A19. A system according to any of the preceding embodiments wherein the wellbeing enhancement logic has machine learning ability.

Embodiment A20. A system according to any of the preceding embodiments wherein the wellbeing processor provides data quantifying at least one aspect of an end-user's wellbeing to a secured remote data repository and wherein the system includes a remote processor which learns how to enhance wellbeing based on data arriving from multiple counterparts of the wellbeing processor and wherein the remote processor repeatedly configures the wellbeing enhancement logic in at least the wellbeing processor as the remote processor learns.

Embodiment A21. A system according to any of the preceding embodiments wherein the processor detects an activity which the end-user is engaged in and the wellbeing enhancement logic defines how to control the at least one digitally controlled home appliance when an end-user is engaged in the activity.

For example, if the processor detects that the end-user is sitting or reading, the logic may define that shades or shutters in a room in which the end-user is sitting and/or reading, should be adjusted to reduce sunlight apt to cause glare. Or, if the processor detects that the end-user is exercising, the logic may define that the air conditioner's thermostat should be lowered and once the exercise session terminates, the logic may define that the air conditioner's thermostat should be raised.

Embodiment A22. A system according to any of the preceding embodiments wherein the processor detects the activity which the end-user is engaged in at least partly based on the sensors (such as a wearable pulse or heartbeat sensor, identifying heightened pulse or heartbeat, or an accelerometer detecting rate of movement, any of which or any suitable combination of which may be indicative of exercise).

Embodiment A23. A system according to any of the preceding embodiments wherein the processor detects the activity which the end-user is engaged in at least partly based on the processor's knowledge of the current time combined with the processor's knowledge of likelihood of a given activity taking place at that time, for the end-user or for a group to which the end-user is known to belong.

Embodiment A25. A system according to any of the preceding embodiments wherein the sensors measure at least one of end-user temperature, ambient temperature, humidity, ambient light, pulse, heart rate.

Embodiment A26. A computer program product, comprising a non-transitory tangible computer readable medium having computer readable program code embodied therein, the computer readable program code adapted to be executed to implement a smart home control method comprising using at least one hardware processor to perform:

generating an initial set of recommendations for, and/or operative limitations on, home control actions;

measuring wellbeing indexes aka scores and using the scores as feedback to determine which home control actions improve the scores including
introducing perturbations of device operation including applying at least one perturbation to at least one parameter of at least one home appliance;
measuring the perturbations' effect on the scores, and
further optimizing of house management to yield increased wellbeing by retaining post-perturbation values of at least one individual parameter of at least one individual home appliance, which yielded increased wellbeing relative to pre-perturbation values of the individual parameter of the individual home appliance.

Embodiment A27. A system according to any preceding embodiment wherein the wellbeing enhancement logic comprises a hardware processor which is configured to perform: generating an initial set of recommendations for, and/or operative limitations on, home control actions; and measuring wellbeing indexes aka scores and using the scores as feedback to determine which home control actions improve the scores including introducing perturbations of device operation including applying at least one perturbation to at least one parameter of at least one home appliance; measuring the perturbations' effect on the scores, and further optimizing of house management to yield increased wellbeing by retaining post-perturbation values of at least one individual parameter of at least one individual home appliance, which yielded increased wellbeing relative to pre-perturbation values of the individual parameter of the individual home appliance.

Also provided is processing circuitry comprising at least one hardware processor and at least one memory and configured to perform at least one of or any combination of the described operations or to execute any combination of the described modules.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program, such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities e.g. within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g. digital signal processor (DSP), microcontrollers, field programmable gate array (FPGA), application specific integrated circuit (ASIC), etc.) and other electronic computing devices. Any reference to a computer, controller or processor is intended to include one or more hardware devices e.g. chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface or other system illustrated or described herein. Any suitable computerized data storage, e.g. computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein which may for example include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus the term user interface or “UI” as used herein includes also the underlying logic which controls the data presented to the user e.g. by the system display and receives and processes and/or provides to other modules herein, data entered by a user e.g. using her or his workstation/device.

### BRIEF DESCRIPTION OF THE DRAWINGS

Example embodiments are illustrated in the various drawings. Specifically:

FIGS. 1-2 are simplified semi-pictorial semi-block diagram illustrations of certain embodiments, including possible system components, all or any subset of which may be provided.

FIGS. 3a-3b taken together, and FIGS. 4a-4b, taken together, illustrate respective tables useful in understanding certain embodiments.

FIG. 5 is a simplified semi-pictorial semi-block diagram illustration of an embodiment, including possible system components, all or any subset of which may be provided.

FIG. 6 is a simplified flowchart illustration of a method constructed and operative in accordance with certain embodiments.

In the block diagrams, arrows between modules may be implemented as APIs and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML.

Methods and systems included in the scope of the present invention may include any subset or all of the functional blocks shown in the specifically illustrated implementations by way of example, in any suitable order e.g. as shown. Flows may include all or any subset of the illustrated operations, suitably ordered e.g. as shown. Tables herein may include all or any subset of the fields and/or records and/or cells and/or rows and/or columns described.

Computational, functional or logical components described and illustrated herein can be implemented in various forms, for example, as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology), or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if and as appropriate, and which may employ any suitable architectural option, such as but not limited to FPGA, ASIC or DSP, or any suitable combination thereof.

Any hardware component mentioned herein may in fact include either one or more hardware devices e.g. chips, which may be co-located, or remote from one another.

Any method described herein is intended to include within the scope of the embodiments of the present invention also any software or computer program performing all or any subset of the method's operations, including a mobile application, platform or operating system e.g. as stored in a medium, as well as combining the computer program with a hardware device to perform all or any subset of the operations of the method.

Data can be stored on one or more tangible or intangible computer readable media stored at one or more different locations, different network nodes, or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use; and which is based on any suitable technologies such as semiconductor, magnetic, optical, paper and others.

### DETAILED DESCRIPTION OF CERTAIN EMBODIMENTS

The following terms may be construed either in accordance with any definition thereof appearing in the prior art literature or in accordance with the specification, or to include in their respective scopes, the following:

User—may include any organism staying in a smart home: e.g. a tenant, visitor, or pet.

User wellbeing or health condition—may include a set, group or collection of all biometric parameters acquired on the user, e.g. a point in the multidimensional space of a user's biometrics.

User state—may include the user's situation/activity, e.g. stand, sit, walk, run, etc.

Device—may include smart home apparatus with adjustable properties and output.

CDP—may include Controllable Device Parameter: a parameter in a physical device that the user (and system) can modify remotely, e.g. light intensity, light source color e.g. CCT, media volume, HVAC temperature set, etc.

Scene—may include a specific set of values of the home devices for a specific user at specific time and user state. This set of parameters indicates the user activity and the appliance-parameters e.g. CDPs selected/optimized for this activity.

Physical parameters—may include any parameter retrieved from the house by the system or from public data on non-living objects, e.g. temperature, humidity, light intensity.

Biometrics—may include any biological related parameters of the user, measured by the system.

Thermal comfort—a term of the art, which, according to Wikipedia, refers to a condition of mind that expresses satisfaction with the thermal environment and may be affected by external factors like temperature and air velocity, personal factors like clothing isolation, and maybe even colors and sounds.

Smart home—may include a system that connects home appliances (devices) to a central hub from which they are controlled and monitored. Usually, a set of sensors in the house also collects data on temperature, humidity light level, open/closed doors (e.g., by magnetic field presence or any other suitable sensor) etc.

System designer—may include a person who programs the smart home system, typically by configuring baseline scenes and rules for the basic comfort of the user.

For example a simple graphical user interface (GUI) may be provided which represents 4 typical scenes, “Away”, “Sleep”, “Leisure” and “Active”. All scenes have a dedicated menu item choice which the user can press for activating the scene. An additional input option/menu choice/virtual button may be provided in the user interface, for accessing additional internal settings. According to an embodiment, pressing the virtual button may pop up a password request screen preventing unwanted user access to internal settings. When a correct password or user code (which may be assigned only to special users) is entered, a new menu system is displayed and the user can select menu items such as, say, which appliances may take part in or may influence or may be influenced by a certain scene and/or drill down to sub-menu items including appliance parameters such as, say, min-max range, default settings etc.

In this example, the “Away” scene is when no tenants are inhabiting the house, “Sleep” scene is when the tenant is resting or napping, “Leisure” scene is for the case when a tenant is occupied with some leisure activity as watching TV or reading a book and “Active” scene is when a tenant is engaged with some physical work or sports (e.g., treadmill). These scenes represent respective rules and configuration settings and these “internal” settings may or may not be transparent to the end-user (e.g. may be set by skilled workforce or other special users or super users e.g. as described above). In other cases additional menu choices are present for specific settings (e.g., by pressing A “*” menu item). For example, the “Away” scene may call for shutting off any climate control systems while the “Active” scene may call for setting the climate control systems to maximum performance. the internal settings behind each scene may be adjusted or perturbated by a central controller over time (i.e., over time the system learns that the minimum temperature settings should not drop below 20 degrees due to specific tenant preferences).

Example: the system starts at a “Sleep” scene (may represent the tenant resting). The tenant then wakes up for some sport activity and changes the scene manually to “Active”. A corresponding message may be displayed on the screen indicating that the system has confirmed the manual transition to this new state. Some time afterwards the system may automatically decide that the tenant is away (e.g., based on some presence measurements) and switches automatically the scene to “Away” and a corresponding message is displayed. While the system may be right in most cases, a correction option may be available to the user to change the automatic decision which may have been wrong due to various factors (e.g., algorithm accuracies etc.). for example, perhaps the user overrides the system's decision to “Leisure”. Such manual input from the end-user may be used to retrain (e.g., re-enforcement) system scene detection algorithms.

More generally, according to certain embodiments, the system may include a GUI (graphic user interface) which defines plural states or modes e.g. all or any subset of: active, leisure, sleep and away. Any suitable rules may be provided to govern transitions between modes. For example, the user may select that sh/e is now entering a certain state such as the “active” state. Or, the system may detect that the user has transitioned to another stage e.g. that the user is away. The system may have rules about what state to be in if the system detections contradict the user's inputs. For example, the user may be entitled to override the system and command the system to transition to, say, the leisure state.

CS—Circadian Stimulus: may include a metric known in the art for measuring the effectiveness of a light source in providing circadian stimulus, which ranges from 0 (no stimulus) to 0.7 (full saturation). This stimulus activates hormonal response that regulates the “biological clock”.

ppm—Parts per million

Ppb—Parts per billion

PM—Particulate matter. General air pollutants are classified by their size. PMX are airborne particles in the air with a diameter of X microns (m−6) or less. PM10, PM2.5, PM1 are the main pollutants monitored.

CCT—Correlated Color Temperature: may include a measure of light source color appearance defined by the proximity of the light source's chromaticity coordinates to the blackbody locus.

Indexes—may include any computed score for a group of biometrics related to one objective, e.g.: sleep (comprised of sleep regularity, snoring, duration etc.), rest (comprised of: rest heart rate, breathing rate, body temperature etc.). For example, if the sleep score index is only a function of the number of “wake-ups” N and the accumulative “snoring” time Ts over the complete sleeping duration T, a suitable function may be:

$$SleepScore = \frac{1 - a ln b}{{log}_{2} ( N + 2 ) - a ln ( b + \frac{Ts}{T} )}$$

Where a,b are predefined score coefficients. If N=0 (no wake ups) and Ts=0 (no snoring) then the sleep score is 1. If either N>0 or Ts>0 the sleep score may decrease. In addition, if a=0 then only the wake up events are influencing the sleeping score while if we set a=1 and b is set to a small value such as b=0.1 then the snoring periods become more dominant. It is appreciated that systems which monitor snoring are known (e.g. https://www.sleepassociation.org/sleep-disorders/snoring/snoring-apns/#:˜:text=Snore%20Lab%20(iOS%20and%20Android.your%20results%20in%20the%20morning

Wellbeing score—may include a weighted sum of plural available biometrics. The score is typically normalized and aimed at representing the general wellbeing or health status, as can be inferred from the acquired biometrics. The actual weights can be prefixed according to some research based significance (e.g., the value of biometric A may receive a higher weight than the value of biometric B if research indicates that B is, relative to A, less significant). The weights may be updated over time due to research updates and/or individual habits that the system may have learned. For example, certain individuals' wellbeing are highly sensitive to their quality of sleep in which case the “quality of sleep” biometric index may be weighted higher for such individuals, than for other individuals whose wellbeing is less affected by quality of sleep. It is appreciated that sleep quality metrics are known, e.g. https://www.ncbi.nlm.nih.gov/pmc/articles/PMC6223557/#:˜:text=The%20single%2Ditem%20sleep%20quality%20scale%20(SOS)%20is%20a.standards%20of%20sleep%20quality%20evaluation.

To learn how to compute wellbeing, e.g. by properly weighting available biometrics, a system may at least initially use a known wellbeing assessment tool e.g. as described here: https://www.hpft.nhs.uk/service-users/recovery/tools-for-change/or here https://bmcgeriatr.biomedcentral.com/articles/10.1186/1471-2318-9-55 or here http://www.fepto.com/wp-content/uploads/Validity-of-the-Well-Being-Scale.pdf to determine how best to predict assessed wellbeing, by combining the available biometrics.

The frequency of updates may vary as appropriate (e.g., tied to software updates and/or to convergence times of algorithms tracking tenant habits etc.).

It is appreciated that references herein to wellbeing may according to certain embodiments be replaced by references to wellness or by references to health. Typically, wellbeing includes mental, emotional, and physical components.

ML—Machine learning.

Artificial Intelligence (AI)—may include any device that which takes actions that maximize its chance of successfully achieving its goals.

### Tib=Time in bed

### WUT—Wake-Up Time

### SI=Number of Sleep Interferences

Ap=Apnea events
Sd=Sleep depth
SHR=Sleep heart rate
ExF=Excrement frequency

### BW=Body Weight

SS=Stool shape
ST=Stool texture
SB=Stool hidden Blood

### SM=Stool Microbiome

Sdy=Stool density
UC=Urine chemistry parameters
SN=Steps number
Ca=Calories burnout
Ci=Calories intake
Coc=Caffeine consumption

### BT=Body Temperature

### BR—Breathing Rate

RHR: Rest Heart Rate (awake)
maxHR: maximal Heart Rate (awake)

### AHR: Activity Heart Rate.

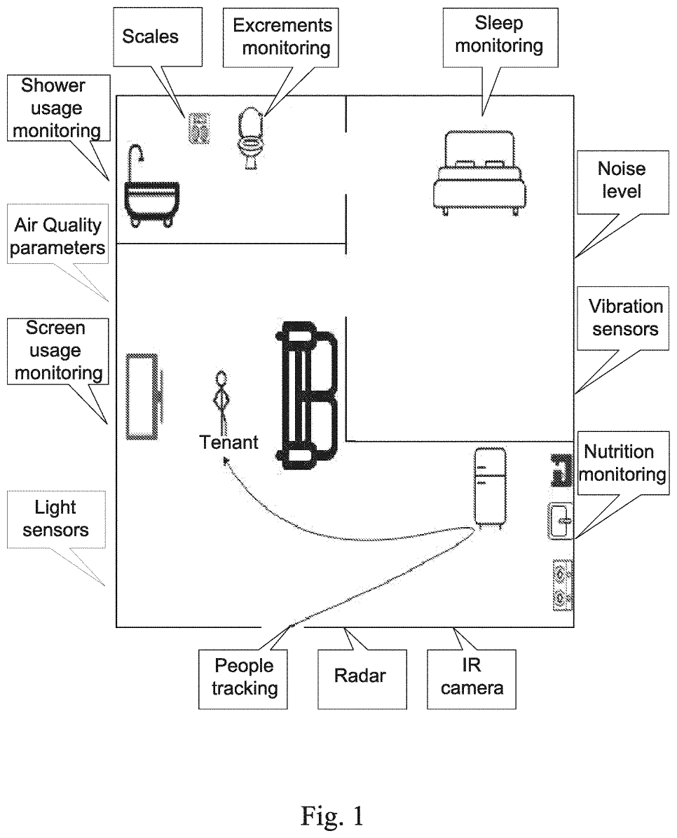

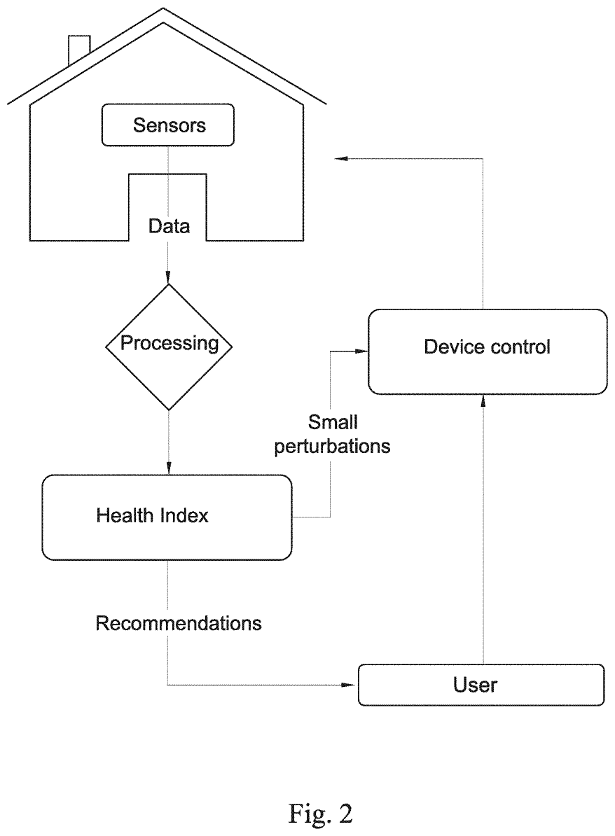

System hardware may include environmental sensors for physical parameters, such as but not limited to all or any subset of temperature, humidity, light intensity, light source color e.g. CCT, etc. located in relevant locations in the house; and wellbeing sensors e.g. sensors for biomedical parameters such as but not limited to heart rate and sleep parameters located in relevant locations in the house (e.g. sleep quality below the bed, heart rate in the living room, body temperature in the living room, etc. and sensors that can indicate the user state (like a radar or IR imaging device which may be installed, say, on the room ceiling or on the walls). For example, FIGS. 1-2 are simplified semi-pictorial semi-block diagram illustrations of certain embodiments, including possible system components, all or any subset of which may be provided. In FIG. 2, health indices and/or wellbeing indices may be used.

The system typically builds itself a data structure e.g. by collecting data from all or any subset of the following data sources:

Data source 2a: Appliance-setting e.g. CDP—a setting on home devices which are controllable, and their value level could be retrieved e.g. via an “Internet of Things” setup (e.g. light fixture intensity, light fixture color e.g. CCT, shades, air flow etc.).

Data source 2b: Public data, relevant to the home operation and user decision making, for example weather, sun positioning, external air quality and other external sources or conditions whether geographical, climate region etc.

Data source 2c: Default scenes included in the smart home system software and scenes entered by the user.

Data source 2d: Biometric sensors distributed in the house in plural positions e.g. for collecting real time data of a specific house inhabited by specific individual/s.) Data source 2e: Biometric optimal data from peer reviewed scientific literature, or public average.

Data source 2f: Medical based rules for improved health and wellbeing, entered by the system designer, based and updated from peer reviewed scientific literature.

Data source 2g: Threshold level for each appliance-setting e.g. CDP, entered by the system designer.

Data source 2h: Logs of all appliance-parameters e.g. CDP values, system computations and operations.

Data source 2i: Wellbeing individual effect score, e.g. as computed in operation d described herein and quantify the effect of appliance-parameter e.g. CDP (e.g. appliance-setting) modifications on individual user's wellbeing score.

An algorithm (aka main flow) performed by the system herein (which may take effect after an initial period of operation of the house, e.g. for 6 weeks, or more, or less) may include all or any subset of the following operations a-g, in any suitable order e.g. as shown:

Operation a: On each new house related input, use MUAI to infer the current scene (e.g. away or sleep or leisure or physically active) or refer to the scene indicated by the user.

Operation b: Compute, determine or select a relevant appliance-parameter e.g. CDP (e.g. appliance-setting) that should be changed (by applying a perturbation to that parameter/setting) in order to improve user's wellbeing or health.

Operation c: During the next activation of the scene, decide (as described below) whether to apply the appliance-parameter e.g. CDP modification or propose a modification to the user and execute a decision.

Operation d: Measure the effect on the wellbeing score by computing the correlations between appliance-parameter e.g. CDP change and wellbeing score parameters (form logs). Consider manual correction applied by the user as a factor that can override wellbeing effect (reinforcement learning).

Operation e: Correct (positive or negative score) the modification done in operation c according to the wellbeing individual effect score computed in operation d.

Operation f: compute wellbeing score every time an input parameter is changed.

Operation g: When further modifications to appliance-parameters e.g. CDPs do not affect the wellbeing score significantly (since the algorithm has reached optimum CDP values), execute the Monte Carlo method by using random changes to related appliance-parameters, e.g. CDPs to avoid local minimum traps.

### Infrastructure

Typically, multiple sensors are distributed in the house for measuring biometrics, as well as physical parameters (e.g. using any of the environmental sensors or wellbeing sensors described herein). The sensors may include: radar (an RF sensor that can detect individual location, size, position, movement, heart rate, heart rate intervals, breathing, blood pressure and more), IR (thermal parameters), acoustics (noise/sound sensors), light intensity, vibration, user weight, UV, temperature and humidity, etc.

Data from all sensors are logged (see e.g. using data source 2a), stored, and processed, to provide vital, timed, information on the house conditions and tenants/pets' behavior and activity. The house e.g. the example house shown in FIG. 1, may be controlled, in the sense that most devices are connected to a main hub (e.g. OpenHab (https://www.openhab.org/)) and may be programmed, remotely controlled, or operated manually. Among the controlled devices are: lights, blinds, door locks, humidifier/dehumidifier, heating, ventilation, and air conditioning (HVAC or other temperature control system), air flow rates, etc. For each device there may be several controllable parameters which are referred to as Controlled Device Parameter/s (CDP/s). For example, a light fixture (Device) could have two appliance-parameters e.g. CDPs: Light intensity and light color (CCT).

### Automatic Operation of Devices

Information on house physical parameters, tenants' biometrics and state, are collected continuously from multiple sensors. Wellbeing score may be computed as described below and monitored continuously (see e.g. using data source 2i). Appliance-parameters e.g. CDPs are maintained to facilitate the optimal environment for maximizing the wellbeing score of the tenants, in specific scenes, using machine learning and AI (see operation b). Some examples for machine learning algorithms are Support Vector Machines, Neural Networks, Decision Trees, Bayesian Networks etc. Each of these are described as having a “optimization goal”, for example the system gets values of heart rate, position in the house and posture, and can classify the exact scene (reading, eating, resting, sleeping, etc.). If a reading scene is detected in the afternoon, the system can react by changing light parameters values (intensity, light source color e.g. CCT), shades position, temperature, etc. These changes may influence the user's sleep quality a few hours later. After reaching the optimum (e.g. appliance settings which are optimal, at the population level, for population wellbeing) computed from population statistics published in peer reviewed papers these settings are also personalized by adjusting them to maximize the user wellbeing score, e.g. using the optimization-by-perturbation functionality shown and described herein.

Typically, the system conducts both a long term process and perturbation methods and short term processes which are used to adapt more quickly to radical changes in the environment or initialization phases (e.g., system turn on for the first time). Perturbation methods are typically slow to converge as they rely on a series of relatively small adjustments. However, the system can typically quickly adjust to new scenarios by either initializing from a non-zero state or by model simplification. A non-zero state typically relies on previous interactions with the system which led to certain operational choices. For example, configuring a room for optimal sleep may include at least some parameters which are invariant to the exact room location such as climate control parameters hence whatever was learnt about a tenant in one room can be reused for the same tenant in another room. In many other cases, even if the tenant scenario is completely new, some parameters can be set according to general population data or by averaging out the parameters of other tenants set within the same scenario. Using Model simplification (e.g. as described herein https://ieeexplore.ieee.org/document/8248126) parameters may be disregarded for an initial period and once the system stabilizes, these additional parameters are re-introduced. For example, room location for initialization may be disregarded and later on the room location can be added taking into account for example external light sources which may exist due to a certain room direction.

More generally, any model simplification technique may be employed to yield more rapid convergence providing the system with the ability to offer a specific service such as, say, configuration of light intensity and/or spectrum (color) or configuration of preferred climate control without being encumbered by an overly long initial orientation period or waiting time. This is particularly useful for cases in which a user changes her or his habits suddenly e.g. happens to go to sleep at 5 pm rather than 10 pm or suddenly decides to start exercising daily in the living room rather than, as previously, in the bedroom.

Example: Configuration of preferred climate control may for example comprise configuring fan air flow level or temperature.

Typically, initial settings are configured which are optimal according to a given current state of knowledge e.g. given available historical data from any source, regarding optimal settings of, say, temperature.

Since optimization of the wellbeing score is a function of several inputs that may contradict one another, the system typically uses multi-objective optimization methods, e.g. the Bellman equation. An example of multi objective optimization is a reading scene. Light and/or thermal comfort constraints may be factored in, when deciding on optimum opening level of the shades. The Bellman equation is but one example of a multi-objective optimization method.

According to certain embodiments, the system learns the specific need of each user for each activity by analyzing the historical data, while taking into account user previous preference in controlling appliance-parameter e.g. CDP (a.k.a. behavior), physical parameters, and relevant public data (e.g. using data source 2b). Further optimization can be achieved by making small perturbations on appliance-parameter e.g. CDP while monitoring the effect on the wellbeing score and correcting the modification if the user has rejected the change by manually changing the modified appliance-parameter e.g. CDP (see operation d). It is appreciated that manual changes as used herein are intended to include any override of a current setting determined by the system, whether by the end-user e.g. elder herself or himself, or because the end-user called into a center, and, responsively, human support staff or an automatic artificially intelligent service changed the current, system-determined setting.

While appliances may have some internal parameters which are available for configuration through various means (e.g., communication link, user GUI panel, etc.) it is sometimes the case that less than all appliance parameters may be directly accessed. For example, some climate control systems may include a fan element which can operate at various speeds, but may not enable fan speed control through its remote and the speed may change only as a result of an internal control process while trying to achieve certain climate conditions. In this case the end user may have access to a physical or virtual knob or slider (possibly as part of a GUI panel) which can be set to various levels between “hot” to “cold” and the internal controller may translate these choices to exact settings for temperature and fan speed parameters.

For example, small perturbation of reading scene's Appliance-parameter e.g. CDP can be reducing the light intensity by 5% or 10%. The reading experience may be the same while the sleep quality later may be improved. The small perturbation may continue, say, until the user explicitly reverses the system action by manually changing the light intensity, or until the wellbeing score is found to not be an improvement. For example, at a subsequent time, in a subsequent perturbation, the light intensity may be re-tuned to a higher level (e.g. to the previous level, before the 5%-10% reduction) or may be further reduced, say by another 5% or 10%. If this further perturbation improves wellbeing, the above small perturbation may be discontinued, and the new level of light intensity (the further perturbation) which is subsequently found to improve wellbeing, may be continued.

It is appreciated that the above example is not intended to be limiting. Alternatively, or in addition, for example, the humidity in a room might be increased by 1%—or by 20%.

it is appreciated that references herein to appliance-parameters may alternatively be replaced by references to appliance-setting.

It is appreciated that optimization may begin from any suitable initial state of appliance settings. For example, each setting may initially be set at a middle setting (e.g. shutters may initially be half closed, or a “medium” setting, for an appliance having three levels of intensity (low/medium/high), or at a setting which is believed, due to prior research or a priori knowledge, to be optimal e.g. 60% humidity might be optimal for a particular population. For example, in an IOT setup in which each appliance's settings are collected centrally in a data repository, each appliance may be set initially to a level which is believed to be optimal, such as a central tendency (mean or median of mode) setting for a given population of tenants, or for a given subpopulation (e.g. women, elders, residents of city x, residents of north-facing apartments, living room vs. kitchen/bedroom etc.) to which a given tenant and/or appliance are known to belong. It is appreciated that research is available e.g. visual comfort probability (VCP), also known as Guth Visual Comfort Probability, is a known metric defined as the percentage of people that are happy with a certain lighting scene (viewpoint and direction), and do not find it causes visual glare. Other glare ratings include The Unified Glare Rating (UGR), the Visual Comfort Probability, and the Daylight Glare Index.

According to certain embodiments, at least some appliances are periodically re-initialized centrally. For example, the air-conditioner may be re-initialized from a heating setting to a cooling setting, for all tenants in a multi-tenant facility, on 1 June, each year, and may again be re-initialized, from the cooling setting back to the heating setting, on 1 October, each year. Or, especially in an Internet-of-Things setup, tend analysis may allow more sophisticated central re-initialization. Alternatively, the system allows the system herein, via applying perturbations, to transit to the settings appropriate for each season, without any central re-initialization. It is appreciated that time series prediction is a known predictive modeling problem. The Long Short-Term Memory network or LSTM network is a type of recurrent neural network which can cope with sequence dependence and can be developed, say, in Python using e.g. the Keras deep learning library. Kalman filters may be used for time series analysis, to make estimates of a current state, and recommend responses accordingly. In a first, prediction operation, the Kalman filter may estimate current state variables and associated uncertainties. When an outcome (which may include random noise) of a subsequent measurement is available, estimates are updated using say a weighted average, with estimates having higher certainty being assigned higher weights.

It is appreciated that the optimization yielded by applying perturbations as described herein, is at the level of the individual end-user or tenant. Typically, although not necessarily, the optimization-by-perturbation process is active continuously. For example, perturbations may be introduced to each of a few parameters in each of three appliances, such as lighting, shutters, air conditioner, in each of a few rooms of a given tenant. After each perturbation, the system typically retains the new (post-perturbation) setting if wellbeing has improved as a result of the perturbation, and typically restores the appliance to the old (pre-perturbation) setting, otherwise. As soon as this has been done, the cycle repeats, and perturbations are again introduced, initially, say, again to the lighting, and then to the shutters and air conditioner, initially in the kitchen, say, and then in the bedroom and living room.

Typically, perturbations are random in direction (e.g. some randomly selected perturbations may increase humidity whereas others may decrease humidity) in order to aim for and converge or drift to a global optimum operational point.

Wellbeing optimization, e.g. further optimization, typically comprises improving a given group of settings, typically for all appliances in a residence, typically, although not necessarily, one setting at a time, by following a suitable process of optimization-by-perturbation, such as the following process: introducing a perturbation in an individual setting (transitioning an individual setting of an individual appliance e.g. from a first level to a second level, determining the person's wellbeing, comparing the person's wellbeing to her or his wellbeing before the perturbation was introduced, and determining whether the perturbation is associated with a positive or negative effect on the person's wellbeing. If the effect was positive (but typically not otherwise), the setting of the appliance at the second level may be maintained, and, typically, perturbation of another setting in the same appliance or another appliance, is introduced, or, a further perturbation is introduced in the same setting e.g. transitioning the individual setting of the individual appliance from the second level to a third level. It is appreciated that if the second level was higher than the first level, the third level is selected to be higher than the second level, and vice versa: if the second level was lower than the first level, the third level is selected to be lower than the second level. As time goes on, this process slowly optimizes the settings. Due to possible interactions between the effects of various settings on wellbeing and/or due to fixed or periodic changes in external variables which may confound the effects of various settings on wellbeing, a given perturbation in an individual setting may be re-tested on occasion. For example, various appliances' parameters' settings may be subjected to the above process, and when all appliances and all parameters have been subjected to the above process, the process begins again, such that the optimization-by-perturbation process is active continuously. Fixed changes include for example a new tenant, a change in a given tenant's wellbeing or state of health e.g. the tenant has fallen and has broken her or his hip, or a change in a tenant's lifestyle e.g. a tenant has begun to work out, or has signed up for courses. According to one embodiment, perturbation is applied to only a single setting of a single appliance, and additional settings of additional appliances are only investigated after conclusions have been reached (determinations have been made) regarding results on wellbeing of applying perturbation to the single setting of the single appliance. For example, illumination intensity in a tenant's living room may be increased by 10%, and a conclusion may be reached (a determination may be made) that this improves wellbeing, e.g. because a post-perturbation wellbeing score exceeds a pre-perturbation wellbeing score. In this case, the illumination intensity may again be increased, say by applying a perturbation of another 10%, in the same direction, and a conclusion is reached, say, that this does not improve wellbeing; in this case intensity may be restored to the previous level, and a perturbation is now applied to an additional setting of, say, the air conditioner. Or, as soon as the initial perturbation is shown to improve wellbeing, the intensity level reached after the initial perturbation is maintained, and then the perturbation is applied to, say, the air conditioner.

According to another embodiment, perturbation is applied in parallel to plural settings of a single appliance (e.g., for an air conditioner, settings both for room temperature and for humidity), or to one or more setting/s of each of plural appliances, and multivariate techniques are employed to ascertain whether each setting of each appliance contributed to, or is associated with, increased wellbeing, or decreased wellbeing. Application of perturbation in parallel to first and second appliance settings may include applying perturbation to both appliance settings simultaneously (e.g. at 10 am on 31 May), or applying perturbation to the second appliance setting while a time-period during which effect of the first appliance setting on wellbeing is being evaluated, is still ongoing. For example, each appliance setting's effect on wellbeing may be evaluated over a two-week period, however, perturbations may be applied to seven different appliance settings during that two-week period, once every two days. Two weeks after perturbation was applied to the first appliance setting, the first appliance setting's effect on wellbeing is evaluated by using, say, regression analysis or analysis of variance, to computationally isolate the effect of the first appliance setting, only, on wellbeing (e.g. the percentage of variance in wellbeing which is attributable to the first appliance setting). While application of perturbations is a slow process, the system as described herein typically has model simplification functionality so as to serve end-users reasonably well even when their habits change suddenly, by responding relatively well relatively quickly, and responding even better as the perturbation application techniques lead the system to further optimization.

Typically, wellbeing enhancement logic, stored in computer memory, defines how appliances' modes of operations affect at least one end-user's wellbeing at at least one time t. For example, if incrementing a given appliance's setting from level1 to level2, decrements the user's wellbeing, because the user's wellbeing scores drop rather than rise responsive to the setting transiting from level1 to level2, then the logic may store this fact in any suitable format.

It is appreciated that the length of time over which wellbeing needs to be evaluated, may be learned by the system. For example, it may turn out that whatever effect on wellbeing is gained by increasing humidity, becomes evident only after 10 days, whereas effects of reduced room temperature become evident within only 24 hours.

Any suitable method may be used to determine the person's wellbeing after a perturbation has been introduced. For example, wellbeing may be computed repeatedly e.g. hourly, daily, or weekly during a given time-period beginning when the perturbation was introduced, each time typically by combining several current measurements of several components of wellbeing respectively, yielding plural wellbeing values, and the final determination of the person's wellbeing after a perturbation was introduced may be a central tendency e.g. simple average of the plural values. Or, components of wellbeing such as heart rate, pulse and body temperature may be measured repeatedly during the given time-period, yielding plural values of each component, and a single determination of the person's wellbeing may be made by combining central tendencies e.g. simple averages of each of the wellbeing components.

It is appreciated that a determination of a person's wellbeing may be adjusted to reflect external changes which affect wellbeing but are considered unrelated to the effects of the appliance settings. For example, a determination of a person's wellbeing may be adjusted or discounted to correct for the effects of having been infected by Coronavirus during a period which follows application of a change in appliance settings, e.g. if it is believed that the person's having been infected by Coronavirus is so clearly unrelated to appliance setting as to render any changes in wellbeing due to having been infected by Coronavirus, as artifactual insofar as optimizing appliance settings is concerned.

The system also generates by a rule-based design, implemented by the system designer, lifestyle recommendations (see operation c) based on the wellbeing score, and/or all or any subset of the following:
Sensitivity analysis of single parameters that may increase the score upon change.
Behavior of other users, including in other houses with this system, and the effect on their wellbeing (e.g. using data source 2d). e.g. if light intensity increase at noon is done by natural light (blinds opening) showed improved sleep quality for several users in different houses—blinds opening may be proposed to a different user (who usually increase LED light intensity at noontime with closed blinds and preferred by the multi-objective optimization algorithm described above. Some preferences may be predefined (e.g. based on other individual experiences) and may be modified or deleted according to use. For example, a preference which is infrequently used and/or is frequently overruled by user choices may indicate either low necessity for such a preference or need for modification. New preferences can be added through collective experiences (e.g., users of the system which have performed similar operations).
Medical information as interpreted from peer reviewed scientific papers (e.g. using data source 2f). For example, if the user turns on a light rich with blue color at night, the system can recommend not using this specific fixture at this time of the day, because it may reduce the user's sleep quality. An example recommendation flow is shown pictorially in FIG. 1.

Sensor Data

Data from house sensors as well as tenant biometrics are captured and logged (e.g. using the system's environmental sensors and/or using data source 2a). Additional metrics may be used to compute indexes and scores related to heart performance, sleep quality, activity level and other key features as described below. Typically, these indexes are then used to compute the wellbeing score using any suitable analytical expression or logical or computational method to combine the indices typically into a single scalar. For example, the indexes x₁, x₂, x₃, . . . may be represented in an index vector X=(x₁, x₂, x₃, . . . ) together with a weight vector W=(w₁, w₂, w₃ . . . ) of the same dimension, and the wellbeing score may be computed by a dot (scalar) product W^{T}V

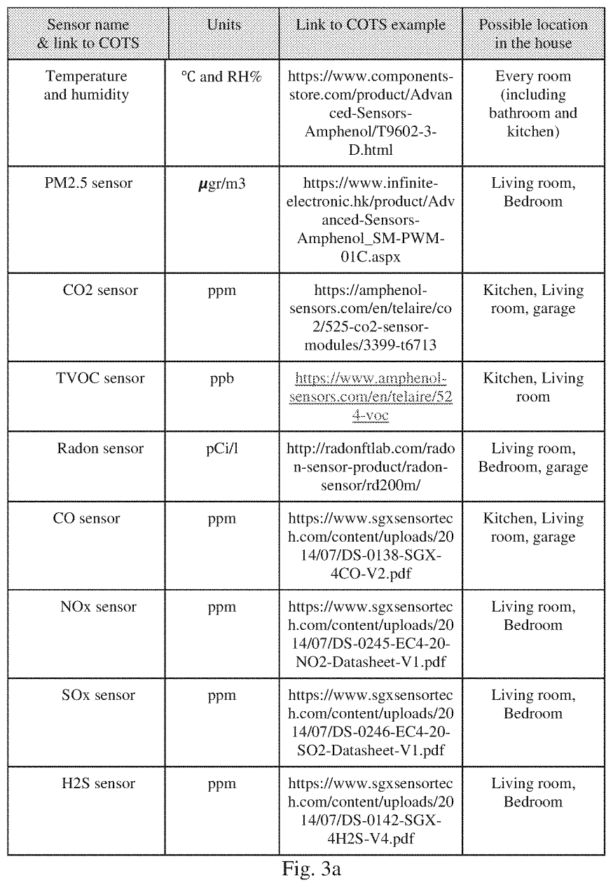

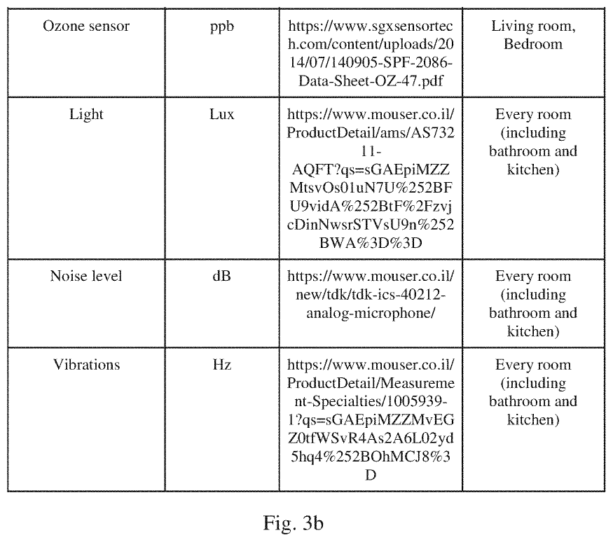

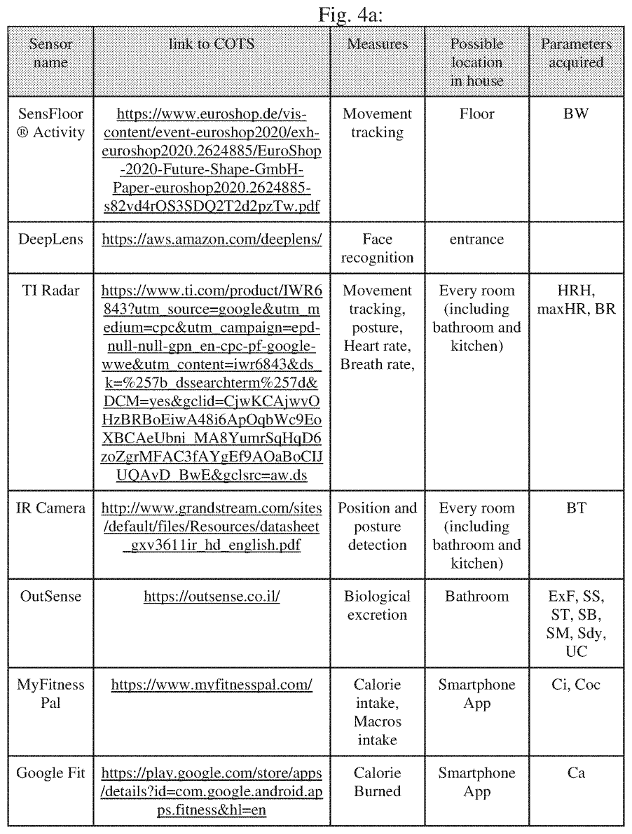

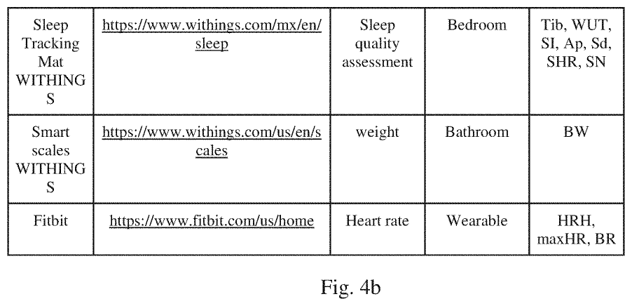

House physical parameters may inter alia include all or any subset (or none) of the entries in the table of FIGS. 3a-3b, taken together. In the table of FIGS. 3a-3b, each sensor may be deployed in any suitable location in a venue e.g. home such as all or any subset of the locations indicated in the rightmost column. It is appreciated that the off the shelf instances of each sensor are provided merely by way of example.

Measurement frequency is typically set for each parameter according to that parameter's rate of potential change. For example, heart rate is measured every 5 seconds since it can change rapidly, and sleep depth is measured every 5 minutes, since sleep cycles are usually longer than 30 minutes. Acquisition location and example COTS is detailed in the table of FIGS. 4a-4b; it is appreciated that all or any subset of the rows and columns and cells of this and other tables may be provided.

The tables herein may be stored as tables in system memory or may be stored in any other suitable format or data structure.

Biometrics may inter alia include all or any subset of the examples listed in the table of FIGS. 4a-4b, taken together. The sensors may be deployed in any suitable location in a venue e.g. house, such as but not limited to all or any subset of the locations specifically indicated in the table by way of example. Parameters acquired by each sensor may include all or any subset of the locations specifically indicated in the table by way of example. Each sensor may measure all or any subset of the specific measurables all or any subset of the locations specifically indicated in the table by way of example.

Computed indexes may include all or any subset of the following:

SRI=Sleep Regularity Index, computed from sleep parameters like, TIB, WUT etc.
Ss=Sleep score example features of Ss, Sleep deepness, REM time, number of waking ups
HR=Heart rate score example features of heart-rate activity HR, RHR Sleep HR
MS=movement score (from house tracking data and fitness apps)
ExS=Excrements score example features of ExS, urine color,

### AQI=Air Quality Index

An example for data structure and processing is now described (e.g. using data source 2d, 2e, 2f). Typically, system data is arranged in vectors, where each vector arranges or stores logically related data together; vectors may include all or any subset of the following:
1. Vm (e.g. user wellbeing vector)—Vector for each timestamp, collecting Biometric measured data in a predefined order (e.g Vm=[80, 37.7, . . . ] represents the measured heart-rate, measured body temperature, etc.).
2. Vo—The optimal biometric parameter value for the specific gender and age (together defined here as user class) of each user in the predefined order (e.g Vo=[76, 37.2, . . . ] the optimal heart-rate, temp, etc. for the user class. If a certain value is not determined in the literature, the Value N/A may appear.
3. Vp—The population average biometric parameter value for each user class. These values are used only if the Vo value is N/A.
4. Vmin—Biometric measured data minimal possible value in a predefined order (e.g Vmin=[40, 35.4, . . . ] minimal value of heart-rate and body temperature based on one standard deviation (SD) from the population value.
5. Vmax—Biometric measured data maximal possible value in the predefined order (e.g Vmax=[160, 42.0, . . . ].—maximal value of heart-rate and temp base on one SD from the population value.

Vmin and Vmax are used for anomaly detection and for score normalization and may be defined as the ∓1 or ∓3 std values of the parameter distributions. The values of Vo and Vp are derived from peer reviewed scientific literature and validated by the system designer who is also responsible for updating the system with new literature data and for revising the data once every reasonable time, e.g. after 1 year.

In addition to the proposed vectors described above, all or any subset of the following matrices may be built (e.g. using data source 2g):

Md—Matrix for each home appliance-parameter e.g. CDP containing a threshold level (±distance from optimum) which is set by the system designer to facilitate the decision if perturbation or user recommendation may be used to adjust the optimal device value (5c in the flow and detailed explanation below). This parameter may set the user “involvement level” and control, thus market and customer research are done to determine the value for each appliance-parameter, e.g. CDP. This parameter could also be personalized and adjusted according to personal preference.

Mou—For each user and for each home appliance-parameter e.g. CDP combination, a matrix of Optimum values per User (Mou), of size S×T may be built and stored in computer memory where S is the number of scenes in which the device can take part (e.g. active/leisure/asleep/away; S=4), and T is a number of different times of the day (e.g. round hours, morning/noon/afternoon/evening/night, etc. for each of which an optimal value per user is entered, for each of the S scenes). Each of the S×T cells in this matrix contains an optimal/recommended value for a given scene and a given time of day, and for a given user and home-appliance. Initial values in the matrix cells may be set by the system designer based on data from the literature (e.g. using data source 2c). Upon use and optimization, cell values typically change, yielding personalization. For example, consider bedroom ceiling light as the appliance, which is serving Aisha Jackson. There may be some preset scenes in Aisha's bedroom light Mou matrix. Typically, each row represents a scene e.g., “away”, “active”, etc. which each are associated with different light intensities (e.g. within the range 0-100% of the bedroom light's maximum power) set through the day, for (say) every round hour yielding 24 horizontal entries for each scene or row, say from 6 am to 5 am. The matrix may be, initially, pre-populated with some values. For example, in the matrix row corresponding to the “away” scene all 24 light intensity values throughover the day may be set to zero, while in the matrix row corresponding to the “active” scene the light is turned on at some low intensity starting at sunrise (e.g. at 6 am, the light is on 10% of its maximum power, at 7 am, 30%, at 8 am and until evening 45%, reverting to only 1% at the tenant's normal sleep-time which may be 11 pm and may continue till 6 am such that the light is on at only 1% till 5 am, then abck to 10% at 6 am as described.

During operation the tenant may override these presets. For example, the tenant may want lights on during night time even if “away” or the system may consider different values due to other real time health optimization issues.

The system typically updates the initial matrix setting timeously based on these inputs. For example once a day or occasionally, the system may apply an update at some fixed time by retrieving the preset row value, multiplying the value as retrieved by some decaying factor d<1, adding the overridden row values weighted by (1−d) and setting the result as the new preset value for the next day—hence over time the row values may be adapted according to the specific use. Example computations:

S1ₙ₊₁=dS1ₙ+(1−d)[New Overridden S1 Row Values]

S2ₙ₊₁=dS2ₙ+(1−d)[New Overridden S2 Row Values]

Alternatively, or in addition, a scene as stored in the system thus may include an activity of a user with desired devices settings for that activity. For example, a user can define “reading in the living room at noon” as the following:
Main light at 65% intensity CCT>3500 CS>0.30.
Shades at 70%.
HVAC temperature set at 22° C.
Media center Off
Noise level 40 dB

Vm may be updated whenever a new parameter is measured. Typically, the new parameter measured value together with the current Vm value are used to compute a new updated Vm value (e.g., Vₘ₊₁=ƒ(Vₘ, new parameter measured) hence the new value for Vm may be computed based on the historical Vm values and the new measurement where ƒ is an update function). For example, Vₘ₊₁=aVₘ, +(1−a)[new parameter measured] where 0<a<1. In this case the new updated value may be a weighted and normalized sum of the previous value and the new parameter measured.

The wellbeing score may then be computed accordingly e.g. as per operation 3f. This may occur as new parameter measurements become available and/or every prefixed time period and/or according to an external request or event). -( ). This score computation may be used for evaluating the effect of modifications to appliance-parameter e.g. CDP on the users' wellbeing or health, as reflected in biometrics parameters and indexes like heart-rate and sleep quality.

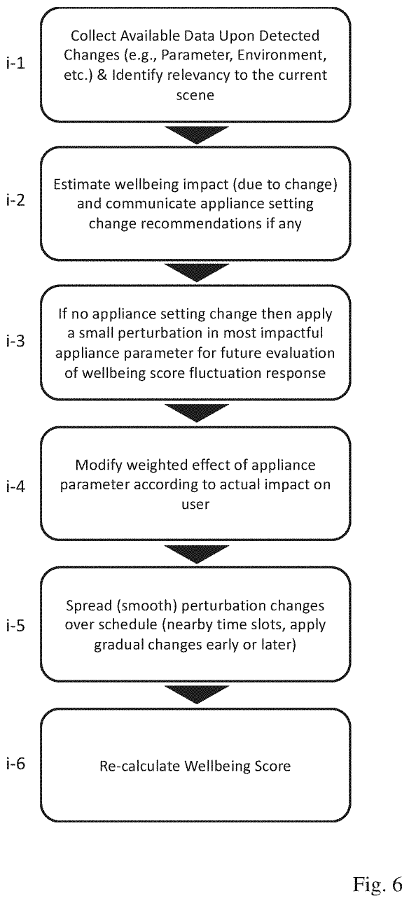

For the system to improve tenant's well-being or health, the method of FIG. 6 may be performed; this method may perform all or any subset of the following operations, suitably ordered e.g. as follows:

Operation i-1. Typically, available data is collected upon detected changes (e.g., Parameters, Environment, etc.) and is correlated to a current scene for identifying relevancy. Whenever a device parameter (or appliance setting) is changed by the user, or for example, whenever a movement is identified in the house, the system may evaluate the current scene, e.g. using a probabilistic model developed by analyzing previous data. This model may take all data as input and, e.g. using artificial intelligence (AI) techniques, finds correlations of specific inputs (specific appliance-parameters e.g. CDPs, day of the week, time of the day, outside temperature, etc.) to specific scenes/activities as defined by the user, or if detected to occur together.

The system may include user location and posture (standing, sitting or lying down) as an input. This data may be collected for example by using a radar system (such as used by cars for measuring obstacles); it is appreciated that many indoor localization radar systems are known such as, for example https://ieeexplore.ieee.org/document/8438759 which describes a radar system for indoor human localization. Also, classification of posture using radar is known; see e.g. https://pubmed.ncbi.nlm.nih.gov/30441089/.

Combining user location and/or posture information typically with data from other biometric and physical sensors facilitates accurate and activity specific identification of scenes. For example, a user lying down, e.g. during night hours, is probably sleeping whereas a user who is sitting, a fortiori standing, is definitely not sleeping.

Operation i-2. estimate most impactful parameter/s which have more impact, relative to other parameters, on the wellbeing score (e.g. due to the changes as detected in operation i-1) and identify desirable appliance setting changes if impact is above a (typically predetermined) threshold. Typically, for the detected scene, in the specific time, the method may list the difference between each appliance-parameter e.g. CDP and its optimal value per person and use the “Wellbeing individual effect score” set initially to 1) to normalize the distance (e.g. by multiplication or division). The normalization process typically provides calibration and impact functions. For example, if one examined appliance parameter leads to a temperature difference value (which can take for example any value between −10 to +10 degrees) and another examined appliance parameter leads to a fan speed difference value (which can take for example any value between −2 to 2), then these differences may be normalized to one scale (e.g., divide the temperature difference by 10 and divide the fan speed difference by 2). Additionally, the method typically weighs-in their actual impact—for example if the temperature difference at a certain time and at a certain operating point has a greater impact (influence or effect) on wellbeing than fan speed difference does, then the temperature difference may be multiplied by a factor A and the fan speed difference may be multiplied by a factor B where A>B to represent A having a greater impact than B. This will eventually translate to the specific effect on the user (initially there may be no effect, but after a few cycles of modifications the system may learn the contribution of each Appliance-parameter e.g. CDP modification on the wellbeing score e.g. as described below). Then, sort the distances in decreasing difference order and choose the value with the largest absolute difference (AD) from the optimum and compare to the threshold value in the matrix Md. In case absolute difference>threshold: propose the optimal value to the user when relevant (e.g. when the same scene/time combination next recurs, or instantly if the duration of the scene allows). Typically, each parameter has a corresponding wellbeing individual effect score whose value may change over time, due to the differences between the estimated effect and the actual effect and/or due to the fact that the effect may increase or decrease as other parameters impact the user's wellbeing.

Operation i-3. When/if the greatest impact (e.g. as computed by operation i-2) is too small (e.g. is below threshold), a perturbation scheme is applied. Typically, a perturbation is introduced, which is typically small enough to be barely noticeable by the tenant, and may be, say, 1-3% of the value of the appliance parameter to which the perturbation is being applied. The size of the perturbation may depend on any suitable factor e.g. the device parameter so for example a 1% perturbation may be applied to one appliance parameter and a 2.5% perturbation may be applied to another, of the value towards the optimum (optimal value) when relevant (e.g. when the same scene/time combination next recurs, or instantly if the duration of the scene allows). In other implementation cases, the perturbation value may be in a random direction in order to avoid sub-optimal convergence. For example, the sign (plus or minus) of the perturbation may be randomly selected.

Operation i-4. The weighted effect of the impactful parameters, typically as detected by operation i-2 and as processed in operation i-3 are now further modified. The effect of each modification on the user's wellbeing score is evaluated, typically. by analyzing logs of the user activity (e.g. using data source 2h), modification implementation and consecutive wellbeing score, and a “Wellbeing individual effect score” (e.g. r2) is given to each modification. If the user did not apply the recommendation, or rejected the modification (e.g. by changing an appliance-parameter e.g. CDP back to the original value) correct the “Wellbeing individual effect score” to reflect the user preference, e.g. by reducing the value by the percentage of change that the user rejected/inverted (see operation d; for example, a modification or perturbation may have increased light intensity by, say, 30% and the user canceled this modification—in this case the method may reduce the “Wellbeing individual effect score” by some corresponding percentage, such as 5%).

Operation i-5. Introduce required changes for optimizing wellbeing, gradually over time. Typically, whenever recommendation is accepted by the user (e.g. is not changed by the user), the values of appliance-parameter which are adjacent in time to the processed change itself are also modified (e.g., CDP in a scenes/time combination in close time slots). For example if “reading at noon” scene\s light intensity was corrected from 200 to 250 lux, adjust the intensity of “reading at 11 a.m” and “reading at 13” to 240, and the intensity of “reading at 10a.m” and “reading at 14” to 230. This may initiate optimization of rarely measured scenes or may ensure that even rarely measured scenes are optimized (e.g. see operation e).

Operation i-6. Re-compute wellbeing score. Typically, after every change in appliance-parameter e.g. CDP, the wellbeing score is measured and go back to operation i-1. Several rounds of this loop may be performed to reach a personalized, optimal setup of appliance-parameters e.g. CDPs in each scene/time combination used. Reaching the optimum may be detected by not being able to change the wellbeing score significantly for a few loops' execution (e.g. five loops in which appliance-parameters e.g. CDPs are modified). In this case the Monte Carlo method may be executed to allow escaping a local minimum; at least one appliance-parameter e.g. CDP may be changed randomly or regardless of the factors computed (e.g. “Wellbeing individual effect score” and “distance from the optimum”). “distance” may be operationalized in any suitable manner and may differ (e.g. over appliances).

For example, in some cases the absolute value of the difference between 2 values can be considered as a distance with some normalization factor but in other cases the square of the difference may be used. In the case of vector distance, a generalized N-power distance between two vectors U,V can be defined by the difference vector D=U−V and then

$$d = \sqrt[N]{\sum {\vert {u}_{i} - {v}_{i} \vert}^{N}} .$$

This may facilitate random walking away from the optimum for cases in which the optimum point found was a local optimum (Optimum trap) (see operation g).

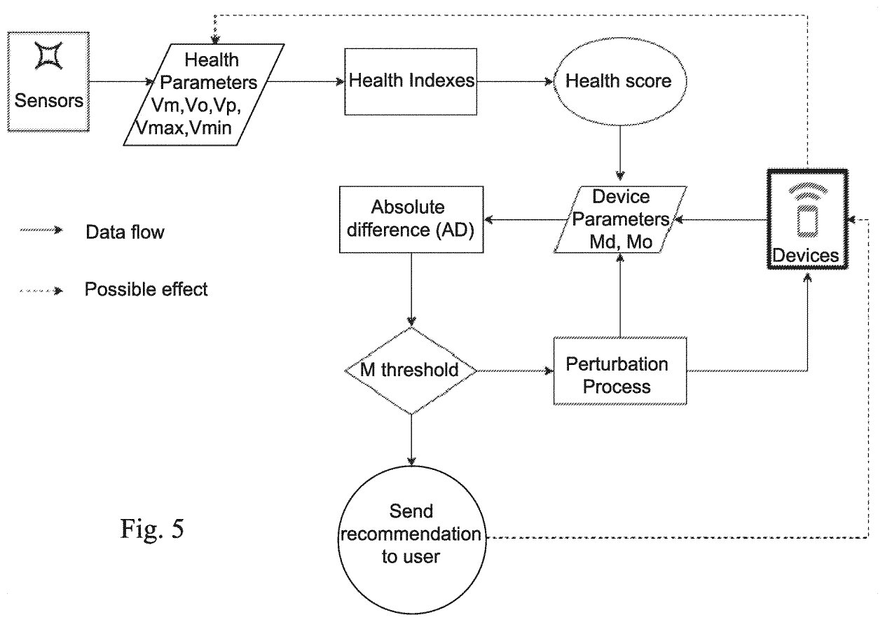

This method facilitates true automatic operation, considering short-term comfort and long-term health and wellbeing, in contrast to the preprogramming requirement in “traditional smart home” systems, e.g. as shown in FIG. 5, the use of data from multiple sources and the usage of human behavior is a significant advantage over current AI based devices that utilize their own operation patterns in order to build prediction models.

This system estimates what the user currently needs (e.g. as per operation 3a), in terms of house control, and thus can also predict what is the most probable next step, and therefore what the user is about to need next. This is an additional implementation of the ML/AI that may be used for home device control. In addition to the devices' operation, useful information is gained by analyzing users' activity. Safety rules are applied to minimize tenants' risk by rule-based design, e.g. if the oven is running, alert when a tenant under a certain age/size is approaching the kitchen without an adult. Manual rule-based design is applied to prevent most of the risk. AI is used to evaluate new/additional risk factors and alert when they tend to recur, and for detecting anomalies, especially in biometric parameters. Security features may be achieved by simple rule-based design, e.g. if someone is detected inside the house, although is not detected entering the house through one of the doors, it may be an intruder. AI is used to achieve more sophisticated security by alerting whenever abnormal behavior is detected.

Personalized automation is achieved by placing a camera for face recognition in specific points in the house. For maintaining privacy, cameras could be placed outside the house at every “legal” entry point (e.g. a door). After camera detection, the radar tracking may use labels to identify each user. Identification validation is done by other sensors in an opportunistic manner. Typically, whenever a user parameter is captured, the system evaluates the probability of false detection. For example, if user A weighing 160 lbs is walking on a hidden scale and detected to weight 200 lbs—the system may evaluate if the tagging system had a false identification and the user is in fact user B, last weighted at 198 lbs.

More generally, it is appreciated that for the purposes of the system herein or any other purpose, a hidden scale is a useful aid for classifying individuals who appear in a given location, where the scale is deployed, as being one or another members of a group such as the group of residents or employees of a venue in which the location is located. For example, a hidden scale may augment face-recognition based classification of individuals, since, typically, the various members of a group each have a unique weight, relative to most or all other members of the group.

Another feature that the system can provide is “averaged/localized comfort”. If more than one user is in a room, the system first tries to adjust heating, ventilation, and air conditioning (HVAC), light and other parameters to fit each of the users. If this is not possible, the system computes the “discomfort price” (e.g. by Biometrics, or by previous responses to similar conditions) and adjusts the parameters such that all users have discomfort to the same extent.

Example for wellbeing score computation:

Vm[1] is the measured heart rate=70 bpm
Vo[1] is the optimal HR (heart rate) value of the specific user (evaluated after one month of measurements)=66 bpm
Vp[1] is the population average of males at the age of 45=64 bpm

### Vmin=40 bpm

### Vmax=240 bpm

Both (Vmin and Vmax) set to eliminate noise and for normalization purposes.
N is the number of measured Biometrics, whereas n is the position of the value in the vector.
Mathematical representation: n=(1,N).
If 21 parameters are measured (as listed above) then N=21 and n can get a value from 1 to 21, while the order of parameters is set and constant.
The score is built by measuring the distance of the measured value Vm[n] from the personal optimum value Vo[n].

Pre_Health_Mes[n]=sqrt{(Vm[n]−Vo[n])\hat{ }2}

If a specific measurement is missing for a specific value, the Vm[n] Value may be taken from the Vp[n] Value.
If Vm is out of the range (Vmin[n],Vmax[n]) the system could be programmed to send a notification to the user.
Each Pre_Health_Mes Value is normalized to a value between 0-1 by the using the Vmin[n] and Vmax[n], e.g. using the following computation:

Pre_Health_Norm[n]={Pre_Health_Mes[n]/(Vmax[n]−Vmin[n])}/N

For this case, no weights are assumed for the different parameters, but such weights could be added.

One example of the wellbeing score formula is:

Health_score=100*[N−Sum(Pre_Health_Norm[n])]/N

The first measurements may be ignored to reduce potential noise. User's specific Vo[n] Value may be computed after a sufficient (e.g. 4 weeks of data acquisition) number of data points are gained.

### ADDITIONAL EXAMPLES

A user is usually reading a book in bed before going to sleep. By computing Circadian Stimulus aka CS level, the system may recommend (by prompting a text message to the user via the home control application for example) changing the light intensity to reduce Circadian Stimulus aka CS. If the effect is not validated (operation d), the recommendation may change to “read out of bed” or “further decrease the intensity”. If the effect is positive, minor perturbations may be applied to further improve the sleep quality, in a seamless manner e.g. using the perturbation process of FIG. 5.
Thermal conditions are adjusted to enable thermal comfort, as defined by the user (comfort set point, not a specific temperature as done in HVAC systems). Plural house parameters (such as radiant flux, target temperature, humidity, air flow and more) could be changed to achieve the same desired set point, and the effect on wellbeing or health is measured. With time and usage, the system measures the wellbeing or health effect of each appliance-parameter e.g. CDP to maximize wellbeing score while reaching the user set point of thermal comfort.

It is known that “Light is the major synchronizer of circadian rhythms to the 24-hour solar day” (https://journals.sagepub.com/doi/abs/10.1177/1477153515592948?journalCode=lrtd). Selecting a suitable architecture to facilitate circadian needs is known .e.g.: “the circadian system requires more light to be activated and is more sensitive to short-wavelength light. Without access to daylight, or electric lighting providing a comparable amount, spectrum, distribution, duration, and timing, human health and well-being may be compromised. This may be particularly true for . . . residents in care facilities. Architectural and design features, including window size, surface reflectances, and furniture placement, impact circadian stimulus levels.” Also, It is known (https://www.lrc.rpi.edu/resources/newsroonmpr_story.asp?id=338#.YD9q2mgzZPY) that “When specifying lighting for the circadian system, it is important to consider light level, spectrum (color), timing and duration of exposure, and photic history (previous light exposures) . . . . Exposure to a CS of 0.3 or greater at the eye, for at least one hour in the early part of the day, is . . . associated with better sleep and improved behavior and mood.”

According to embodiments of the invention, a system such as any system herein is provided which is configured to monitor, directly or indirectly, the amount of light a given human e.g. senior has accessed, given a particular facility with given lighting fixtures, windows, and shades, and to adjust illumination in at least one room in which the senior is spending time, to ensure that the senior is exposed to daylight-comparable illumination of a predetermined cumulative amount at predetermined times of day. The system also typically measures senior wellbeing in order to (a) provide inputs to an MIUAI data system which learns population-level relationships between the amount and/or color and/or timing of light humans are exposed to, and the humans' wellbeing, and/or in order to (b) accumulate individual statistics for a given senior, in order to learn, for this given human, relationships between the amount and/or color and/or timing of light this human is exposed to, and this human's wellbeing.

Any suitable technology may be employed to monitor, directly or indirectly, the amount of light a given human e.g. senior has been exposed to, such as but not limited to receiving data from a wearable ambient light sensor whose geographical position is known, and/or receiving weather data indicative of sunny vs. overcast days, and/or receiving data from light fixtures and/or windows with adjustable shades or transparency whose geographical positions are known and/or receiving navigation data which, when compared to a known architectural plan of a building and/or map of an indoor/outdoor facility, identifies when a given human was indoors and outdoors, and in which room.

It is appreciated that terminology such as “mandatory”, “required”, “need” and “must” refer to implementation choices made within the context of a particular implementation or application described herewithin for clarity and are not intended to be limiting, since in an alternative implementation, the same elements might be defined as not mandatory and not required, or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order including simultaneous performance of suitable groups of operations as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform e.g. in software any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g. by one or more processors. The invention shown and described herein may include (a) using a computerized method to identify a solution to any of the problems or for any of the objectives described herein, the solution optionally include at least one of a decision, an action, a product, a service or any other information described herein that impacts, in a positive manner, a problem or objectives described herein; and (b) outputting the solution.

The system may, if desired, be implemented as a network—e.g. web-based system employing software, computers, routers and telecommunications equipment, as appropriate.

Any suitable deployment may be employed to provide functionalities e.g. software functionalities shown and described herein. For example, a server may store certain applications, for download to clients, which are executed at the client side, the server side serving only as a storehouse. Any or all functionalities e.g. software functionalities shown and described herein may be deployed in a cloud environment. Clients e.g. mobile communication devices such as smartphones may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are if they so desire able to modify the device to obtain the structure or function.

Any “if -then” logic described herein is intended to include embodiments in which a processor is programmed to repeatedly determine whether condition x, which is sometimes true and sometimes false, is currently true or false and to perform y each time x is determined to be true, thereby to yield a processor which performs y at least once, typically on an “if and only if” basis e.g. triggered only by determinations that x is true and never by determinations that x is false.

Any determination of a state or condition described herein, and/or other data generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may, for example, comprise changing the state or condition, or may more generally cause any outcome which is technically advantageous given the state or condition or data, and/or may prevent at least one outcome which is disadvantageous given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art and particularly, although not limited to, those described in the Background section or in publications mentioned therein.

Conversely, features of the invention, including operations, which are described for brevity in the context of a single embodiment or in a certain order, may be provided separately or in any suitable subcombination, including with features known in the art (particularly, although not limited to, those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation and is not intended to be limiting.

Any suitable communication may be employed between separate units herein e.g. wired data communication and/or in short-range radio communication with sensors such as cameras e.g. via WiFi, Bluetooth or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node).

Any operation or characteristic described herein may be performed by another actor outside the scope of the patent application and the description is intended to include apparatus whether hardware, firmware or software which is configured to perform, enable or facilitate that operation or to enable, facilitate or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawings, and/or elements in the written description may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element e.g. operation described herein may have all characteristics and attributes described or illustrated herein, or according to other embodiments, may have any subset of the characteristics or attributes described herein.

1. A smart home control method comprising using at least one hardware processor to perform:
generating an initial set of recommendations for, and/or operative limitations on, home control actions;
measuring wellbeing indexes aka scores and using said scores as feedback to determine which home control actions improve said scores including
introducing perturbations of device operation including applying at least one perturbation to at least one parameter of at least one home appliance;
measuring said perturbations' effect on said scores, and
further optimizing of house management to yield increased wellbeing by retaining post-perturbation values of at least one individual parameter of at least one individual home appliance, which yielded increased wellbeing relative to pre-perturbation values of said individual parameter of said individual home appliance.

2. A method according to claim 1 which stores a matrix of home devices controllable parameters, wherein a threshold level (mth, ±distance from optimum) is given for each parameter to determine whether perturbation or user recommendation are used to adjust the optimal device value.

3. A method according to any of the preceding claims wherein for each home device's controllable parameters, a matrix of S×T (aka S×T matrix) is provided where S is a scene in which the home device aka appliance can take part, and T is a time of day and wherein each vector in said matrix contains an optimal/recommended value.

4. A method according to claim 3 wherein initial optimal/recommended values are given based on prior knowledge whereas, upon use and optimization process, said initial optimal/recommended values may be changed to allow personalization.

5. A method which executes a classification algorithm to classify tenant aka end-user aka user state.

6. A method according to claim 5 wherein classes of tenant state include at least one of: sleep, active, resting.

7. A method according to claim 5 or 6 wherein, for at least one given state, indexes relevant for that state are computed.

8. A method according to claim 3 wherein for a given scene/time combination, the parameters are sorted in decreasing difference order and, at least once, a value with a next highest absolute difference (AD) from the optimum is selected.

9. A method according to claim 8 wherein at least once, an optimal value is proposed to a tenant when the given scene/time combination next recurs.

10. A method according to claim 9 wherein said optimal value is proposed if absolute difference (AD)>Mth.

11. A method according to claim 8, wherein at least once, a perturbation of the value towards an optimum is introduced, when the given scene/time combination next recurs.

12. A method according to claim 11 wherein said perturbation is introduced if absolute difference (AD)<Mth.

13. A method according to any preceding claim wherein a penalty procedure is used, including moving to a next parameter on a list of parameters sorted in decreasing difference order, if a tenant reverses an automatic perturbation.

14. A method according to claim 3 or 8-12 wherein a smoothing procedure is used in which, for every parameter changed, after validation of wellbeing effect, an adjacent time interval is changed to apply change to scenes close in time in the S×T matrix thereby to initiate optimization of rarely measured scenes.

15. A method according to any preceding claim wherein Monte Carlo method is used to produce randomness, thereby to avoid local minimum traps.

16. An automated system for optimizing quality of life of tenants, the system comprising:
wellbeing enhancement logic, stored in computer memory, which defines how appliances' modes of operations affect at least one end-user's wellbeing at at least one time t;
a hardware processor (aka wellbeing processor) in data communication with said logic;
sensors which are configured to generate at least one measurement of at least one aspect of an end-user's wellbeing and to feed said measurement to said hardware processor; and
smart home apparatus including at least one controller which controls at least one digitally controlled home appliance;
wherein the hardware processor is configured to command the at least one controller to at least once control said at least one digital appliance to transit from a first mode of operation to a second mode of operation at a time t, wherein the second mode of operation serves the end-user's wellbeing at time t better than the first mode of operation does, according to said wellbeing enhancement logic.

17. A system according to claim 16 wherein the home appliance comprises a light fixture, shades, an air conditioner, a humidifier, or a window.

18. A system according to any of the preceding claims 16-17 wherein at least one of the sensors is wearable by the end-user.

19. A system according to any of the preceding claims 16-18 wherein said wellbeing enhancement logic has machine learning ability.

20. A system according to any of the preceding claims 16-19 wherein said wellbeing processor provides data quantifying at least one aspect of an end-user's wellbeing to a secured remote data repository and wherein the system includes a remote processor which learns how to enhance wellbeing based on data arriving from multiple counterparts of said wellbeing processor and wherein the remote processor repeatedly configures said wellbeing enhancement logic in at least said wellbeing processor as the remote processor learns.

21. A system according to any of the preceding claims 16-20 wherein the processor detects an activity which the end-user is engaged in and the wellbeing enhancement logic defines how to control said at least one digitally controlled home appliance when an end-user is engaged in said activity.

22. A system according to any of the preceding claims 16-20 wherein the processor detects the activity which the end-user is engaged in at least partly based on said sensors (such as a wearable pulse or heartbeat sensor, identifying heightened pulse or heartbeat, or an accelerometer detecting rate of movement, any of which or any suitable combination of which may be indicative of exercise).

23. A system according to any of the preceding claims 16-20 wherein the processor detects the activity which the end-user is engaged in at least partly based on the processor's knowledge of the current time combined with the processor's knowledge of likelihood of a given activity taking place at that time, for said end-user or for a group to which said end-user is known to belong.

25. A system according to any of the preceding claims wherein said sensors measure at least one of end-user temperature, ambient temperature, humidity, ambient light, pulse, heart rate.

26. A computer program product, comprising a non-transitory tangible computer readable medium having computer readable program code embodied therein, said computer readable program code adapted to be executed to implement a smart home control method comprising using at least one hardware processor to perform:
generating an initial set of recommendations for, and/or operative limitations on, home control actions;
measuring wellbeing indexes aka scores and using said scores as feedback to determine which home control actions improve said scores including
introducing perturbations of device operation including applying at least one perturbation to at least one parameter of at least one home appliance;
measuring said perturbations' effect on said scores, and
further optimizing of house management to yield increased wellbeing by retaining post-perturbation values of at least one individual parameter of at least one individual home appliance, which yielded increased wellbeing relative to pre-perturbation values of said individual parameter of said individual home appliance.

27. A system according to claim 16 wherein said wellbeing enhancement logic comprises a hardware processor which is configured to perform:
generating an initial set of recommendations for, and/or operative limitations on, home control actions;
measuring wellbeing indexes aka scores and using said scores as feedback to determine which home control actions improve said scores including
introducing perturbations of device operation including applying at least one perturbation to at least one parameter of at least one home appliance; measuring said perturbations' effect on said scores, and
further optimizing of house management to yield increased wellbeing by retaining post-perturbation values of at least one individual parameter of at least one individual home appliance, which yielded increased wellbeing relative to pre-perturbation values of said individual parameter of said individual home appliance.
